(19) United States
(12) Patent Application Publication
(10) Pub. No.: US 2006/0064169 A1
(43) Pub. Date: Mar. 23, 2006

(54) Shock-absorbing joint and spine replacements

(76) Inventor: Bret A Ferree, Cincinnati, OH (US)

Correspondence Address: GIFFORD, KRASS, GROH, SPRINKLE & CITKOWSKI, P.C, PO BOX 7021, TROY, MI, 48007-7021, US

(21) Appl. No.: 10/526,993

(22) PCT Filed: Sep. 10, 2003
(86) PCT No.: PCT/US03/28424

§ 371 Date: Mar. 7, 2005

Related U.S. Application Data
(60) Provisional application No. 60/409,623, filed on Sep. 10, 2002.
(60) Provisional application No. 60/412,209, filed on Sep. 20, 2002.
(60) Provisional application No. 60/416,338, filed on Oct. 4, 2002.
(60) Provisional application No. 60/416,379, filed on Oct. 4, 2002.

(51) Int. Cl. A61F 2/32; A61F 2/30; A61F 2/44; A61F 2/38; A61F 2/36

(52) U.S. Cl. 623/17.12; 623/23.41; 623/17.13; 623/20.28; 623/22.15; 623/23.17; 623/23.63

(57) ABSTRACT

Numerous joint replacement implant embodiments including a total knee replacement implant including a femoral component (102) having a wheel (104); and a tibial component (106) including a shock-adsorbing component with a piston assembly (110) and spring (112). Said implants contain a cushioning or shock-absorbing member to dampen axial loads and other forces. In many embodiments, fluid is force rapidly from the device wherein compression and dampening is achieved by valves or other pathways that allow for a slower return of the fluid back into the implant as the pressure is relieved.

### FIELD OF THE INVENTION

This invention relates generally to prosthetic implants and, more particularly, to artificial disc and joint replacement components incorporating shock absorbers, cushioning mechanisms, and other improvements.

### BACKGROUND OF THE INVENTION

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component. Although the use of resilient materials has been proposed, the need remains for further improvements in the way in which prosthetic components are incorporated into the disc space, and in materials to ensure strength and longevity. Such improvements are necessary, since the prosthesis may be subjected to 100,000,000 compression cycles over the life of the implant.

The same is true of total joint replacements, which must endure repeated compressive stresses associated with daily activities such as walking, running, exercising, sitting and standing. These compressive stresses can eventually cause painful fractures and can often result in the implant loosening after several years. Ultimately, revision surgery may become necessary.

Prosthetic implants that address impact problems are known in the art. For example, U.S. Pat. No. 5,389,107 to Nassar et al. discloses a prosthetic hip implant having an elongate element that extends coaxially from the ball section of the femur component. The elongate element slidably extends into a chamber formed by a tubular insert that is secured in the femur. Contained at the bottom of the chamber is a spring against which the elongate element abuts, thereby providing shock absorption. A pin member extends from the bottom of the chamber and slidably fits into a bore formed in the elongate element. A second spring is disposed between the pin and the bottom of the bore to provide further shock absorption.

U.S. Pat. No. 6,336,941 discloses a modular hip implant that can be custom fit to an individual patient, including a shock absorption system that absorbs compressive stresses that are imparted to the implant. The size of the femoral ball member, size of the femoral stem, femoral neck length, and tension in the shock absorption system are all individually adjustable parameters, depending on the particular patient. A unique coupling member houses a modular spring mechanism that serves as the shock absorber. The coupling member is received into the ball member to an adjustable depth, the adjustment of which varies the length of the femoral neck. The length of the femoral neck can be adjusted during surgery without requiring additional parts.

This invention is broadly directed to spine and joint-replacement components wherein, in preferred embodiments, at least a portion of the respective implant contains a cushioning or shock-absorbing member. Such members, which serve to dampen axial loads and other forces, need not be contained entirely within the joint or disc space, as it may be advantageous according to the invention to provide devices external to the region of direct articulation.

In many embodiments, fluid is forced rapidly from the device with compression, and dampening is achieved by valves of other pathways that allow for a slower the return of the fluid back into the device as the pressure is relieved. In intradiscal configurations, spinal motion occurs by movement of the vertebrae over the device, and by the device changing shape. Various fluids may be used within the device including water or aqueous solutions, triglyceride oil, soybean oil, an inorganic oil (e.g. silicone or fluorocarbon), glycerin, ethylene glycol, or other animal, vegetable, synthetic oil, or combinations thereof. Fluids from the body, such as synovial fluid, may also move into and out of unsealed device components.

In some embodiments, transplanted cells and/or cells plus the extracellular matrix (ECM) or analogues thereof, may be contained in the device. For example, a fluid permeable: fiber bag, carcass as described in my U.S. Pat. No. 6,419,704, or a cylinder or other enclosures as described in my pending U.S. Patent Application Ser. No. 60/379,462 may be used to hold the cells or the cells and ECM within the disc space or elsewhere in the body.

### BRIEF DESCRIPTION OF THE DRAWINGS

FIG. 1 is an anterior view of a total knee replacement (TKR) according to the present invention;

FIG. 2 is a lateral view of the TKR of FIG. 1;

FIG. 3 is drawing of a total hip (THR) embodiment of the present invention;

FIG. 4 shows the use of a membrane used to contain metal or other debris;

FIG. 5 is a cross section of the embodiment of FIG. 4;

FIG. 6 is an axial cross section through the top of the device of FIG. 4;

FIG. 7A is a lateral view of an acetabular component according to the invention;

FIG. 7B is lateral view of the acetabular component of FIG. 7A;

FIG. 8 is a sagittal cross section of the device of FIG. 7A;

FIG. 9A is coronal cross section of another embodiment of the present invention;

FIG. 9B is a coronal cross section of the embodiment of the device drawn in FIG. 9A;

FIG. 10A is a partial coronal cross section of a prosthetic knee including a dampening mechanism; and

FIG. 10B is a partial coronal cross section of the prosthetic knee drawn in FIG. 10A;

FIG. 11 is a lateral view of the spine and a device according to the present invention;

FIG. 12 is an anterior view of the spine and the device of FIG. 11;

FIG. 13 is an axial view of the spine and the device of FIG. 11;

FIG. 14 is a sagittal cross-section of the device of FIG. 11;

FIG. 15 shows an exploded view of the device of FIG. 11;

FIG. 16 is a sagittal cross section of another embodiment of the present invention;

FIG. 17 shows an exploded view of the device of FIG. 16;

FIG. 18 is a view of the lateral portion of the spine and an embodiment with endplate resurfacing components;

FIG. 19 is a view of the anterior portion of the spine and the device of FIG. 18:

FIG. 20 is top view of a three-cylinder embodiment of the present invention;

FIG. 21 is a sagittal cross section of the device of FIG. 20;

FIG. 22 is a sagittal cross section of an embodiment having a single piston;

FIG. 23 illustrates compression of the piston of the device of FIG. 22;

FIG. 24 is an exploded view of the device of FIG. 22;

FIG. 25 is a view of the anterior portion of the device with a low-pressure reservoir;

FIG. 26 is a lateral view of the of FIG. 25;

FIG. 27 is a view inside the device of FIG. 25 the outer membrane in cross section;

FIG. 28 is a full cross section of the device of FIG. 25;

FIG. 29A is a lateral view of a compressed device according to the invention;

FIG. 29B is a lateral view of the device drawn in FIG. 29A after the compression is relieved;

FIG. 30A is a lateral view of a device with a hinge associated with a top endplate;

FIG. 30B is a lateral view of the device with the hinged portion of the upper endplate tilted forward as in spinal flexion;

FIG. 31A is a partial coronal cross section of the spine and another embodiment of the invention;

FIG. 31B is a partial coronal cross section of the embodiment of the device drawn in FIG. 31A;

FIG. 32 is an anterior view of an alternative embodiment of an ADR according to the invention;

FIG. 33 is a coronal cross-section of the spine and the embodiment of a device particularly suited to the L4/L5 level and above;

FIG. 34 is a lateral view of the spine and the embodiment of the device drawn in FIG. 33;

FIG. 35 is an anterior view of the spine, sacrum, and the embodiment of the device drawn in FIG. 33;

FIG. 36 is a sagittal cross section of the spine, sacrum, and the embodiment of the device shown in FIG. 35;

FIG. 37 is a coronal cross section of the spine incorporating a slight variation of the device drawn in FIG. 33;

FIG. 38 is a lateral view of the spine and the embodiment of the device drawn in FIG. 37;

FIG. 39 is a coronal cross section of the spine incorporating a further alternative embodiment of invention;

FIG. 40 is a lateral view of the spine including the embodiment of the device drawn in FIG. 39;

FIG. 41A is a coronal cross section of the spine and yet a further embodiment of the device made of a material with shape memory;

FIG. 41B is a coronal cross section of the spine and the embodiment of the device drawn in FIG. 41A;

FIG. 42 is a coronal cross section of the spine and yet a different embodiment of the present invention;

FIG. 43A is a view of the anterior portion of the spine and another different embodiment of the invention;

FIG. 43B is a coronal cross section of the spine and the embodiment of the device drawn in FIG. 43A;

FIG. 44 is a coronal cross section of the spine and another further embodiment of the invention;

FIG. 45A is a coronal cross section of the spine and yet a different embodiment of the invention;

FIG. 45B is a coronal cross section of the spine and the embodiment of the device drawn in FIG. 45A;

FIG. 46A is a coronal cross section of an alternative embodiment of the present invention;

FIG. 46B is a coronal cross section of the embodiment of the ADR drawn in FIG. 46A;

FIG. 47 is a coronal cross section of an alternative embodiment of an ADR according to the invention;

FIG. 48 is a coronal cross section of the embodiment of the ADR drawn in FIG. 47;

FIG. 49 is a sagittal cross section of a total knee replacement incorporating a device according to this invention;

FIG. 50 is a sagittal cross section of the total hip replacement embodiment of the device of the present invention;

FIG. 51A is a sagittal cross section of a disc embodiment according to the invention;

FIG. 51B is a sagittal cross section an alternative disc embodiment; and

FIG. 52 is a sagittal cross section of an alternative disc embodiment of the invention.

### DETAILED DESCRIPTION OF THE INVENTION

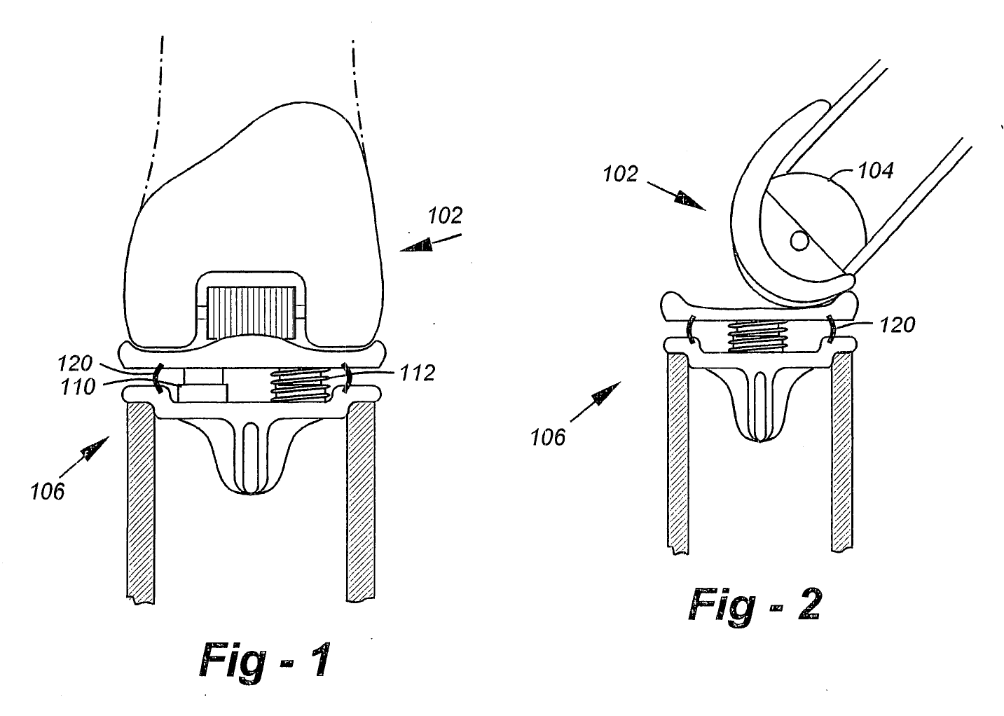

This invention is broadly directed to spine and joint-replacement components wherein, in preferred embodiments, at least a portion of the respective implant contains a cushioning or shock-absorbing member. FIG. 1 is an anterior view of a total knee replacement (TKR) according to the invention. FIG. 2 is a lateral view of the TKR of FIG. 1. The femoral component, 102, includes a wheel 104. The tibial component, 106, includes one or more shock-absorbing components, such as piston assembly 110 and one or more springs such as 112 which may be separate from or surround each piston assembly. The spring on the left in FIG. 1 was not drawn to better illustrate the cylinder halves. An optional membrane 120 may surround the tibial shock absorbers to hold in fluid and/or particulates such as metal debris. The natural synovial fluid within the knee joint may be used to advantage to cooperate with the dampening mechanism.

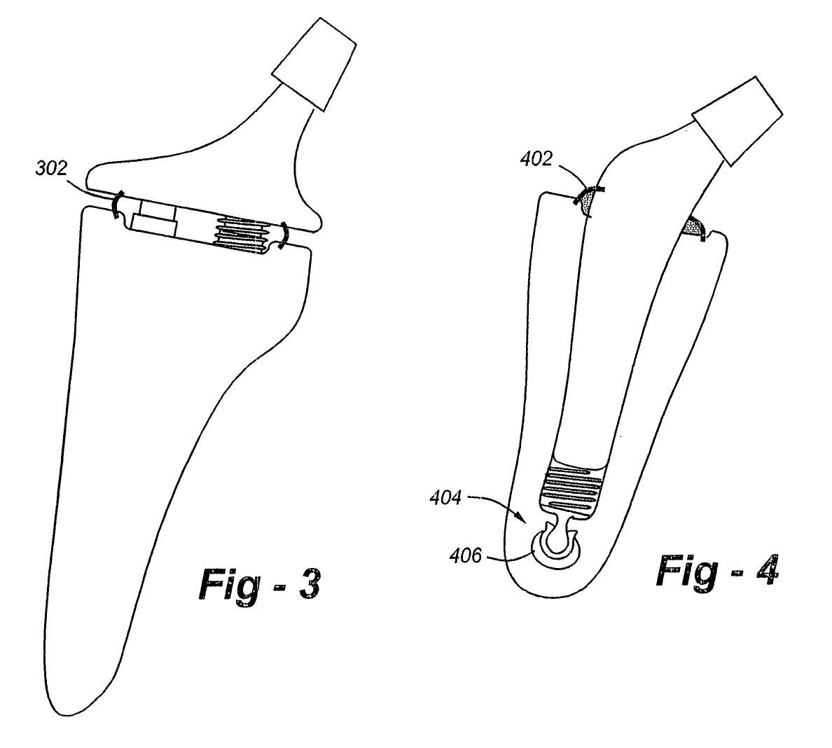

FIG. 3 is a total hip (THR) embodiment of the invention. As with the knee, a membrane 302 may be used to contain fluid and/or debris. The THR could also work on synovial fluid if the membrane were torn or eliminated.

FIG. 4 is an alternative THR embodiment with a membrane 402 and a flap valve 404 below the spring. The expandable membrane surrounding the top of the implant also holds the fluid that is forced through the flap valve. As a further option, the femoral component may contain a space for the inferior membrane to expand into. The inferior space 406 could be closed or contain a hole to communicate with the canal of the femur. Distention of the inferior membrane would compress the air surrounding the membrane. The compressed air would help form the fluid into the area surrounding the spring when tile pressure is decreased.

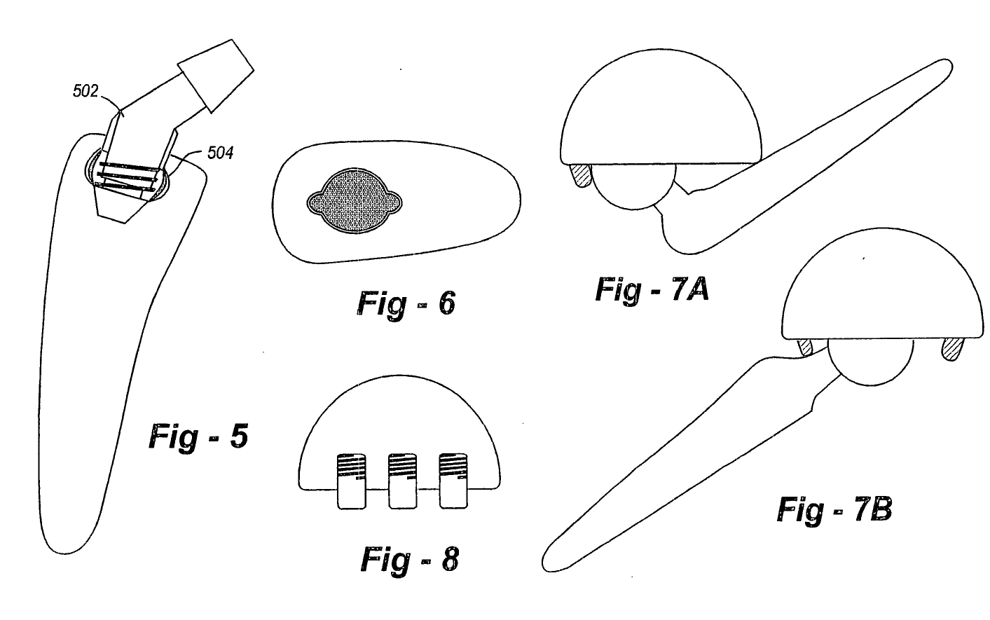

FIG. 5 is a cross section of an alternative embodiment of the invention including a modular, removable shock absorber component 502. FIG. 6 is an axial cross section through the top of the device. Not that the neck of the shock absorber component cooperates with the femoral stem component to prohibit rotation of the one component relative to the other. As in other embodiments, the modular component, contains fluid, a spring and cylinder halves. The femoral stem component has an opening that allows the fluid-containing membrane 504 to expand. A second membrane (not shown) may extend from the base of the (Morse) taper of the shock absorber component to the top of the femoral stem component. The second membrane would contain particle debris generated by pistoning of the shock absorber component within the femoral stem component.

FIG. 7A is a lateral view of an acetabular component according to the invention which includes slidably engageable components that extend from the acetabular component to prevent dislocation. The movable components resist forces perpendicular to the components while reliably collapsing into the acetabular component with axial forces. The posterior movable component resists posterior dislocation of the femoral component. The anterior moveable components collapse in the position shown in FIG. 7A to prevent levering the femoral component out of place.

FIG. 7B is lateral view of the novel acetabulum component of FIG. 7A, with the THR extended. The posterior movable component collapses and the anterior movable components extend in this position of the THR. FIG. 8 is a sagittal cross section of the device. The movable components piston in and out of cylinders in the acetabular component. Springs force the movable components partially out of the acetabular component.

As discussed in the Background of the Invention, in these and in other embodiments, the sealed, fluid containing components may contain water or aqueous solutions, triglyceride oil, soybean oil, an inorganic oil (e.g. silicone or fluorocarbon), glycerin, ethylene glycol, or other animal, vegetable, synthetic oil, or combinations thereof. Alternatively, fluids from the body, such as synovial fluid could move into and out of unsealed embodiments of the device. Indeed, certain configurations according to the invention may use both a sealed fluid and the body's fluid, with seals to prevent the sealed fluid from communicating with the fluid from the body. Pores in a portion of the prosthesis may be sized to permit fluid movement, but to inhibit bone or soft tissue ingrowth into the chamber in the inferior portion of the prosthesis. Wave washers or other spring-like components could be used to force the prosthetic component(s) into an extended, non-compressed position. The stiffness of the spring or springs could vary. Stiffer springs could be selected for heavier or more active patients.

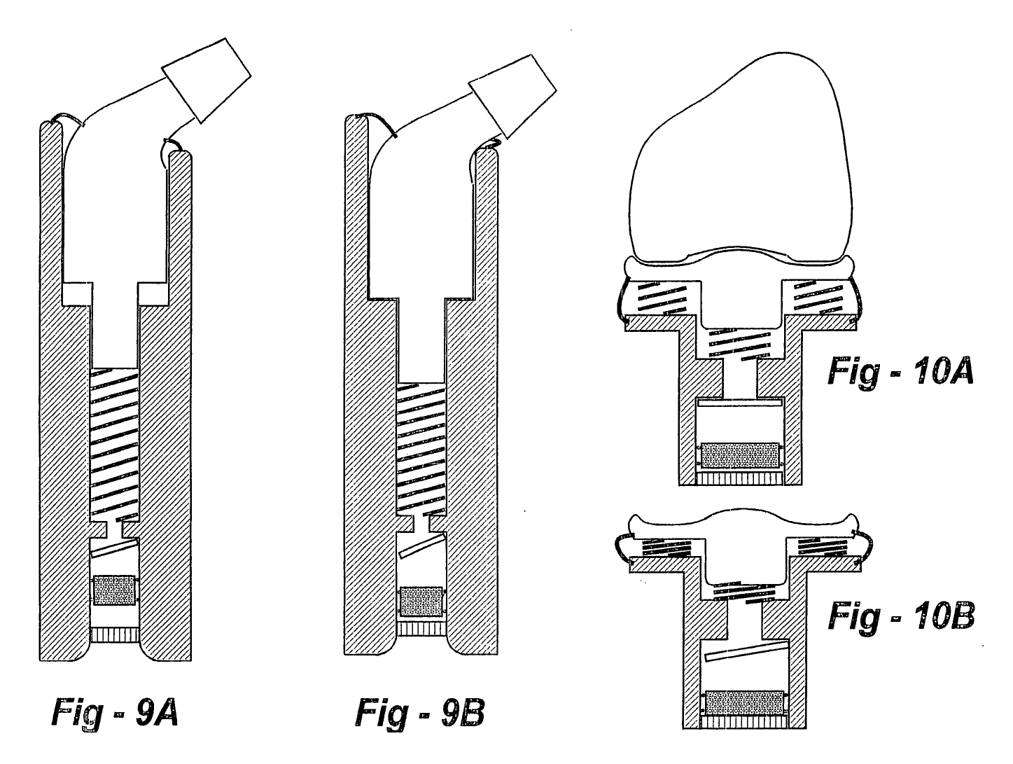

FIG. 9A is coronal cross section of an embodiment including a dampening mechanism associated with a prosthetic hip. A component 902 preferably including a Morse taper pistons in and out of the shaft of the prosthesis. An optional membrane 904 serves to trap debris. The component with the Morse taper may have the anti-rotation features drawn in FIGS. 5 & 6. A piston extending from the inferior surface of the Morse taper component is surrounded by one or more seals, including O-rings seals, to trap fluid within the prosthesis. A spring forces the Morse taper component into an extended, resting, position. A valve such as a flap valve lies below the ledge in the prosthesis that holds the spring. A piston surrounded by seals lies below the valve in a cylinder within the shaft of the prosthesis. A component with pores is seen at the inferior entrance into the cylinder in the inferior aspect of the prosthesis. Fluid moves from the sealed chamber above the valve to the sealed chamber below the valve as pressure is applied to the Morse taper component.

FIG. 9B is a coronal cross section of the embodiment of the device drawn in FIG. 9A. The figure illustrates movement of the components in a reaction to pressure on the top of the Morse taper component. The Morse taper component moves inferiorly, within the shaft of the prosthesis, as pressure is applied to the top of the Morse taper component. Fluid within the upper chamber of the prosthesis is forced through and around the flap valve into the lower chamber as the Morse taper moves inferiorly. The lower piston moves distally within the lower chamber to accommodate the additional fluid. Body fluid below the inferior piston is forced through the porous component and into the shaft of the femur as the inferior piston moves distally. The components of the prosthesis return to the positions shown in FIG. 9A when the pressure is removed from the top of the Morse taper component. The flap valve slows the fluids return to the upper chamber thus dampening the movement across the prosthesis. Fluid from the femur moves into the chamber in the inferior portion of the prosthesis as the inferior piston rises as the sealed fluid moves to the upper chamber.

FIG. 10A is a partial coronal cross section of a prosthetic knee with the novel dampening mechanism illustrated in FIG. 9. The components are drawn in their “extended” position. FIG. 10B is a partial coronal cross section of the prosthetic knee drawn in FIG. 10A. The components are drawn in their “compressed” position. The figure also illustrates the use of an optional expandable membrane used to trap debris within the prosthesis. The figure also illustrates the use of additional springs.

Different aspects of this invention are directed to artificial disc replacement (ADR) devices that use shock absorbers to dampen axial loads across the disc space. Fluid is forced rapidly from the device with compression. Dampening of the axial forces is achieved by valves of other pathways that slow the return of the fluid back into the device as the pressure is relieved. Spinal motion occurs by movement of the vertebrae over the device, and by the device changing shape.

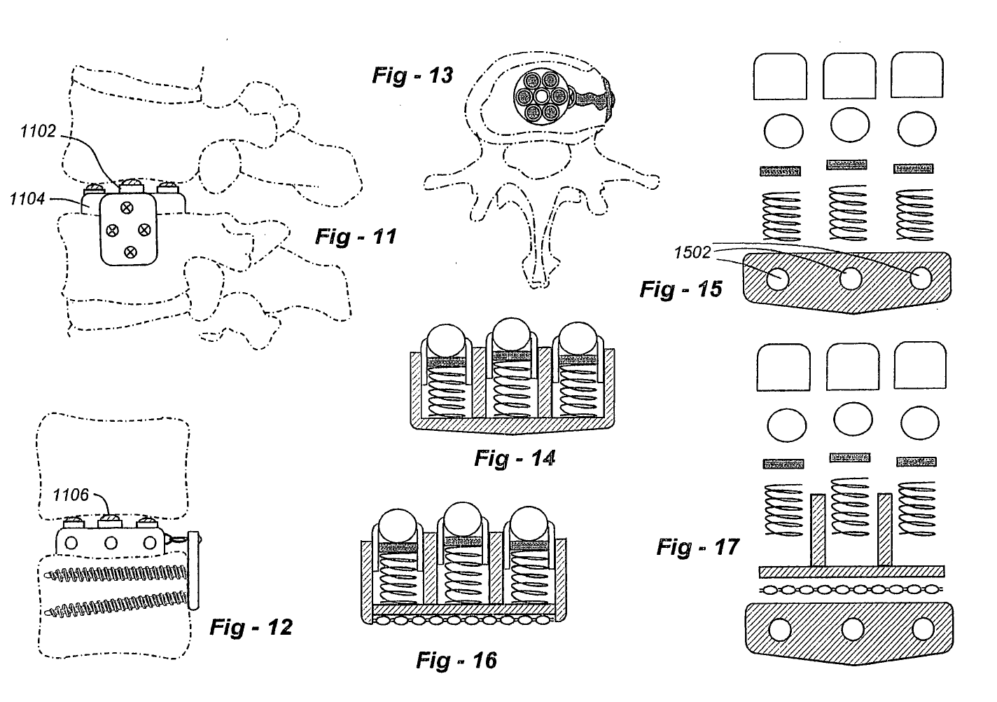

FIG. 11 is a lateral view of the spine and a device according to the invention in position. FIG. 12 is an anterior view, FIG. 13 is an axial view, and FIG. 14 is a sagittal cross-section. Pistons 1102 are housed in cylinders 1104. Springs force the pistons out of the cylinder. This embodiment also shows the optional use of ball bearings 1106 in the pistons. The ball bearings may reduce the friction of the device on the vertebral endplates.

FIG. 15 is an exploded view of the device. The circles 1502 in the body of the device represent valves such as flap valves. The valves allow fluid to leave the device with compression, faster than they allow fluid to return to the device as the compression is relieved. In the preferred embodiment, the device uses natural body fluid. For example, natural lubricant like fluid is frequently found in the joints found in psuedoarthrosis. Similarly, the body frequently manufactures lubricant like fluid to decrease friction between prosthetic devices and overlying soft tissues. In this embodiment the fluid lies freely in and around the device. FIG. 25 shows another embodiment with a fluid containing low pressure reservoir just outside the disc space.

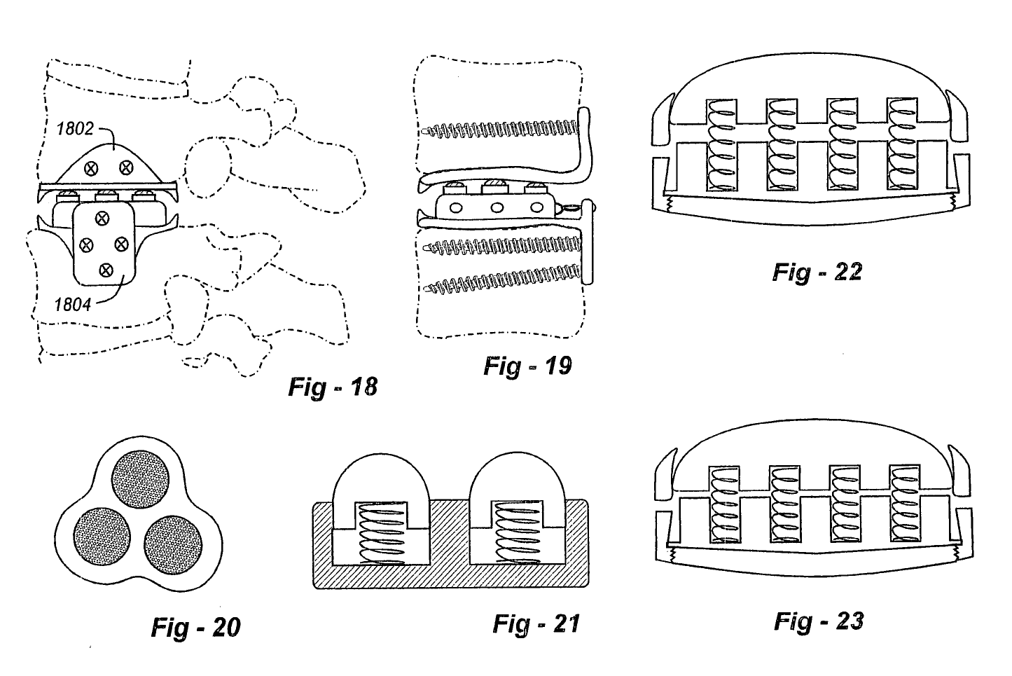

FIG. 16 is a sagittal cross section of another embodiment of the device which incorporates ball bearings on the inferior surface and in the cylinders of the device. FIG. 17 is an exploded view of the device of FIG. 16. FIG. 18 is a view of the lateral portion of the spine and an embodiment with endplate resurfacing components 1802, 1804. The compressible portion of the device is free to move and self-center between the two endplate resurfacing components. The endplate resurfacing components can cooperate to prevent the extrusion of the mobile, compressible member.

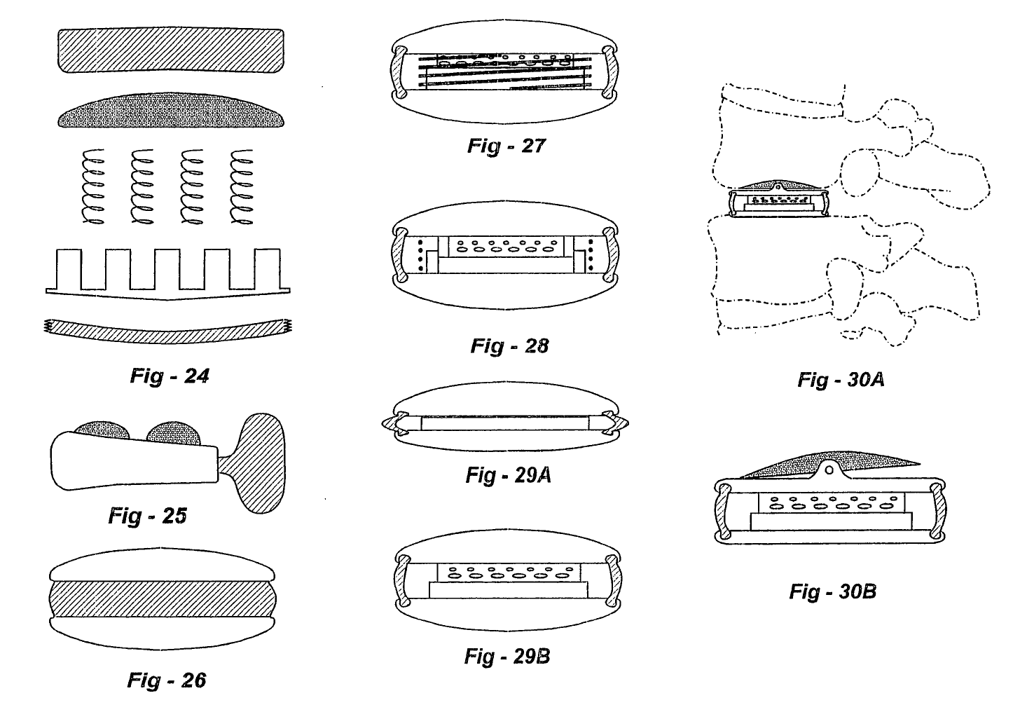

FIG. 19 is a view of the anterior portion of the spine and the device of FIG. 18. FIG. 20 is top view of a three-cylinder embodiment that allows larger pistons, fewer parts, and further exploits the ability of a three-legged structure such as a three-legged stool, to fit very irregular surfaces. FIG. 21 is a sagittal cross section of the device of FIG. 20, also showing an embodiment of the pistons without ball bearings. FIG. 22 is a sagittal cross section of an embodiment having a single piston. FIG. 23 illustrates compression of the piston of the device of FIG. 22. FIG. 24 is an exploded view of the device of FIG. 22. FIG. 25 is a view of the anterior portion of the device with a low pressure reservoir (cross hatched area) that sits just outside the disc space.

The advantages of theses embodiments include the following:

1. Durability. Springs, pistons, cylinders and ball bearings have excellent wear characteristics.

2. The device dampens forces across the disc space. Most ADR designs allow spinal motion. Some ADR designs collapse and expand to accommodate compression forces across the disc space. Few ADR designs dampen axial forces across the disc space.

3. The fluid that moves into and out of the device not only provides dampening of the forces across the disc spaces but also lubricates the moving components of the device.

4. The springs of the device are contained within cylinders to maximize spring function and to prevent the springs from migrating.

5. The compressible portion of the device is mobile to allow the device to self center.

6. The mobile portion of the device is tethered to prevent migration into undesirable locations.

7. The embodiments with ball bearings may reduce the friction between the device and the vertebral endplates.

8. The endplate resurfacing components may decrease the risk of pain from movement of the device over the endplates of the vertebrae.

9. Multi-piston embodiments of the device permit the device to “custom fit” the concavities of the vertebral endplates. The pistons may extend variable distances above this device.

10. The pistons of the multi-piston embodiments of are unlikely to bind. The piston of a single piston device is more likely to bind.

11. Self-centering. One or more components may be attached to a mobile link that allows the ADR to self-center. The device may also be placed between the resurfacing components described above.

FIG. 26 is a lateral view of an alternative ADR including an expandable membrane 2602 that holds fluid within the device. FIG. 27 is a view inside the device with the outer membrane in cross section, and FIG. 28 is a full cross section. A spring surrounds a fluid-filled cylinder. The upper half of the cylinder pistons in and out of the lower half-of the cylinder. Fluid is forced through holes in the upper half of the cylinder with compression of the device. The fluid egresses rapidly at first, through the large holes in the upper half of the cylinder. The fluid exits more slowly as the larger holes in the upper half of the cylinder become covered by the lower half of the cylinder. The fluid that leaves the cylinder is contained within the device by the surrounding membrane. Fluid returns to the cylinder as the device is expanded by the spring urging the cylinder halves apart. Fluid returns to the cylinder slowly at first through the smaller holes exposed initially by the lower half of the cylinder (thus dampening the motion of the device). As the device expands the larger holes in the upper half of the cylinder are exposed, thereby allowing the fluid to return to the cylinder more quickly.

FIG. 29A is a lateral view of the device in a compressed condition. The outer membrane is drawn in cross section without the spring to better illustrate operation. Note that the outer membrane is protruding outward as a result of the endplates becoming closer together and from the fluid moving from the cylinder. FIG. 29B is a lateral view of the device drawn in FIG. 29A after the compression is relieved. FIG. 30A is a lateral view of the device with a hinge associated with the top endplate. The hinge facilitates flexion and ex-tension. The vertebrae are free to move over the device. Tilting of the top endplate allows the vertebrae to flex and extend more with less movement over the device. The upper hinged potion is preferably bi-convex to allow lateral bending. FIG. 30B is a lateral view of the device with the hinged portion of the upper endplate tilted forward as in spinal flexion.

The advantages of theses embodiments include the following:

1. Durability. Springs, pistons, and cylinders have excellent wear characteristics.

2. The device dampens forces across the disc space. Most ADR designs allow spinal motion. Some ADR designs collapse and expand to accommodate compression forces across tile disc space. Few A-DR designs dampen axial forces across the disc space.

3. The fluid that moves into and out of the device not only provides dampening of the forces across the disc space, but also lubricates the moving components of the device.

4. Fewer parts compared to other designs.

5. The compressible portion of the device is mobile to allow the device to self center.

6. The mobile portion of the device is tethered to prevent migration into undesirable locations.

7. The embodiment with the hinged endplate component may reduce the friction between the device and the vertebral endplates.

8. The endplate resurfacing components may decrease the risk of pain due to movement of the device over the endplates of the vertebrae.

As with the joint-replacement embodiments, the fluid containing embodiments may contain water or aqueous solutions, triglyceride oil, soybean oil, an inorganic oil (e.g. silicone or fluorocarbon), glycerin, ethylene glycol, or other animal, vegetable, synthetic oil, or combinations thereof. Alternatively, the expandable membrane of FIGS. 26-31B could be eliminated, allowing fluid from the body to freely move into and out of the ADR.

Wave washers, belville washers, belville springs, or other spring-like components could be used to force the ADR to an extended, non-compressed position. The stiffness of the spring or springs could vary. Stiffer springs could be selected for heavier or more active patients.

The extradiscal portion of the device preferably includes a porous component that allows the body fluid to move in and out of the extradiscal component as the sealed fluid moves in and out of the extradiscal component. The pores are sized to permit fluid movement, but to inhibit bone or soft tissue growth into the chamber of the extradiscal component.

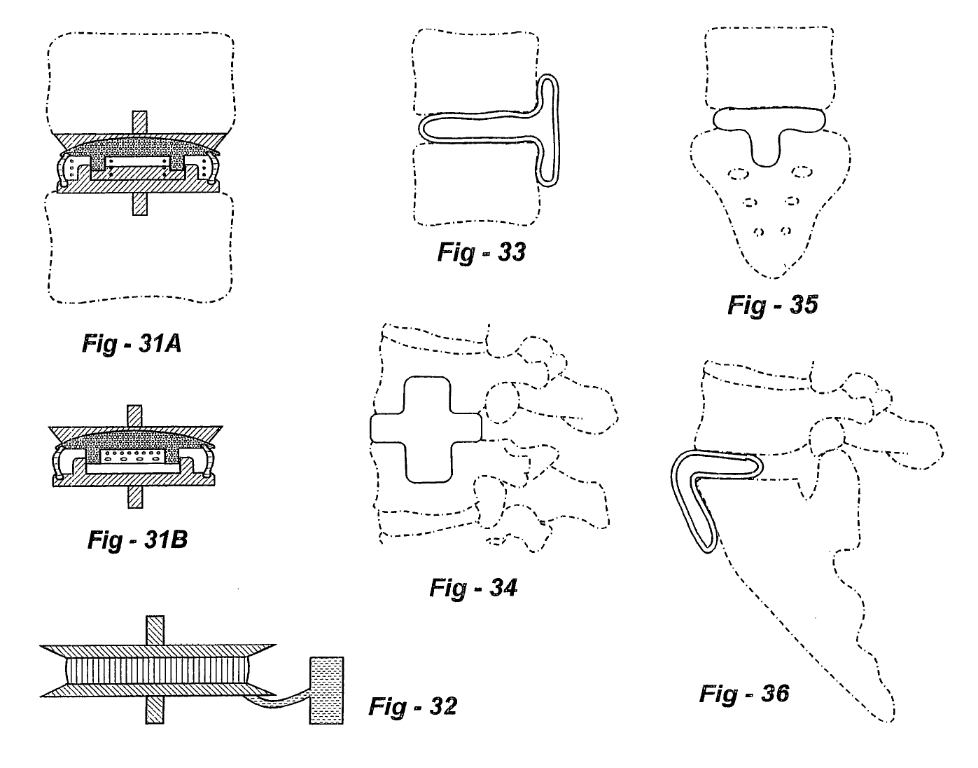

FIG. 31A is a partial coronal cross section of the spine and another embodiment of the device. The bottom of the shock absorbing component is attached to the vertebra below the ADR. The top of the shock absorbing component articulates with an ADR Endplate (EP) that is attached to the superior vertebra. The springs are seen in cross section.

FIG. 31B is a partial coronal cross section of the embodiment of the device drawn in FIG. 31A. The springs were not drawn to better illustrate the inside of the ADR. The figure also illustrates the use of an optional seal between the articulation of the inner cylinder and the outer cylinder. For example, an 0-ring could surround the inner cylinder.

FIG. 32 is an anterior view of an alternative embodiment of the ADR. The embodiment of the ADR drawn in FIG. 31 is connected to an extradiscal component. The outer membrane is preferably flexible, but does not need to be expandable in this embodiment of the device.

Where an extradiscal component is used in conjunction with an intradiscal component, the pressure within the intradiscal component of the device increases as axial loads are applied to the spine or the spine flexes. In operation, fluid within the intradiscal component of the device shifts to the lower pressure extradiscal component as the pressure on the intradiscal component increases. Fluid returns to the intradiscal component of the device as the pressure on the intradiscal component is decreased. Pressure on the intradiscal component is decreased by removing the axial loads on the spine or by returning the spine to a neutral position. The fluid within the relatively high pressure extradiscal component shifts to the lower pressure intradiscal component as the pressure on the intradiscal component decreases. The extradiscal component may be positioned lateral to the spine in from T1-L5 and anterior to the sacrum at L5/S1. The extradiscal component could also be placed at a remote site. For example, the extradiscal component of a cervical ADR could be placed in the chest, or under the skin of the abdomen.

FIG. 33 is a coronal cross-section of the spine and an embodiment of the invention particularly suited to the MA/L5 level and above. FIG. 34 is a lateral view of the spine and the embodiment of the device drawn in FIG. 33. FIG. 35 is an anterior view of the spine, sacrum, and the embodiment of the device drawn in FIG. 33. The embodiment of the device drawn in FIG. 35 is designed for the L5/SI level.

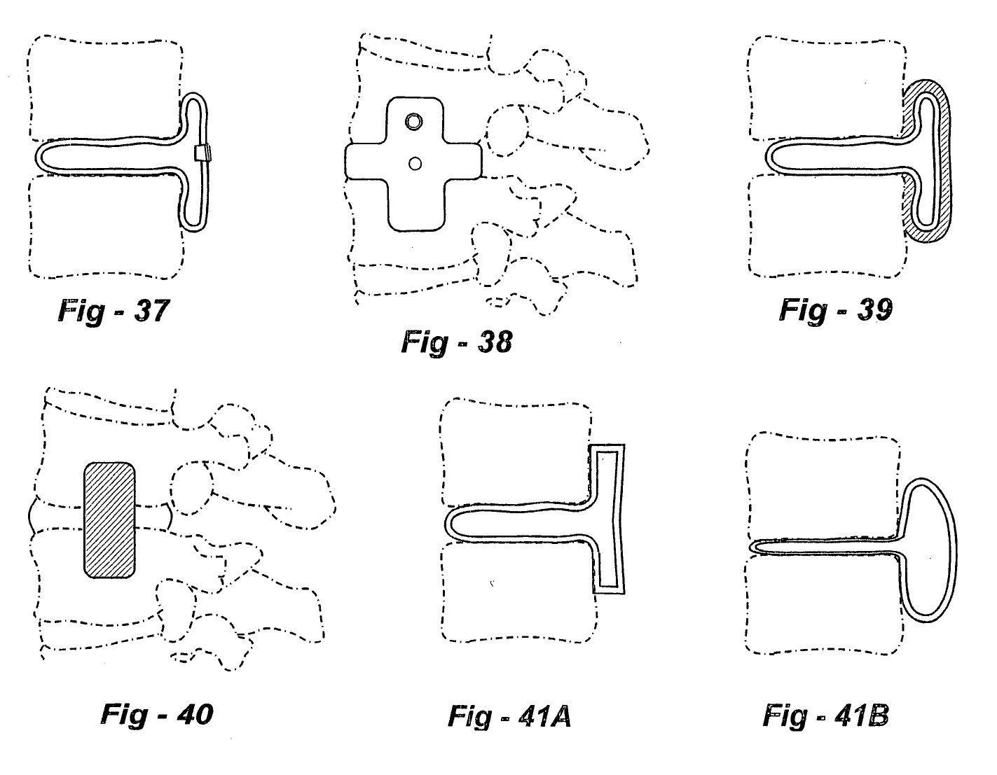

FIG. 36 is a sagittal cross section of the spine, sacrum, and the embodiment of the device shown in FIG. 35. FIG. 37 is a coronal cross section of the spine incorporating a slight variation of the device drawn in FIG. 33, wherein the opening between the intradiscal and extradiscal components is smaller. FIG. 37 also shows the use of a valve to fill the device. FIG. 38 is a lateral view of the spine and the embodiment of the device drawn in FIG. 37.

FIG. 39 is a coronal cross section of the spine incorporating an alternative embodiment of the device. The extradiscal component is surrounded by a sleeve that does not expand. FIG. 40 is a lateral view of the spine including the embodiment of the device drawn in FIG. 39.

The surfaces of each component can be forced from concave to convex or convex to concave if the appropriate forces are applied. For example, the convex intradiscal component becomes concave with the application of axial loads to the spine or spinal flexion. Fluid from the intradiscal portion of the device is shifted to the extradiscal component as the intradiscal component changes from convex to concave. The increased pressure from the shift of fluids forces the concave extradiscal component to become convex. Once the pressure on the intradiscal component of the device is relieved, the extradiscal component returns to its convex shape. The extradiscal component returns to its concave shape. Fluid returns to the intradiscal component as the components of the device return to their preferred shapes.

FIG. 41A is a coronal cross section of the spine and yet a further embodiment of the device made of a material with shape memory. The superior and/or inferior surfaces of the intradiscal portion of the device are preferably convex, whereas the lateral and/or medial surfaces of the extradiscal portion of the device are preferably concave.

FIG. 41B is a coronal cross section of the spine and the embodiment of the device drawn in FIG. 41A. In this Figure, the spine is flexed, the intradiscal component of the device has changed to a concave or flat shape, and the extradiscal component of the device is convex.

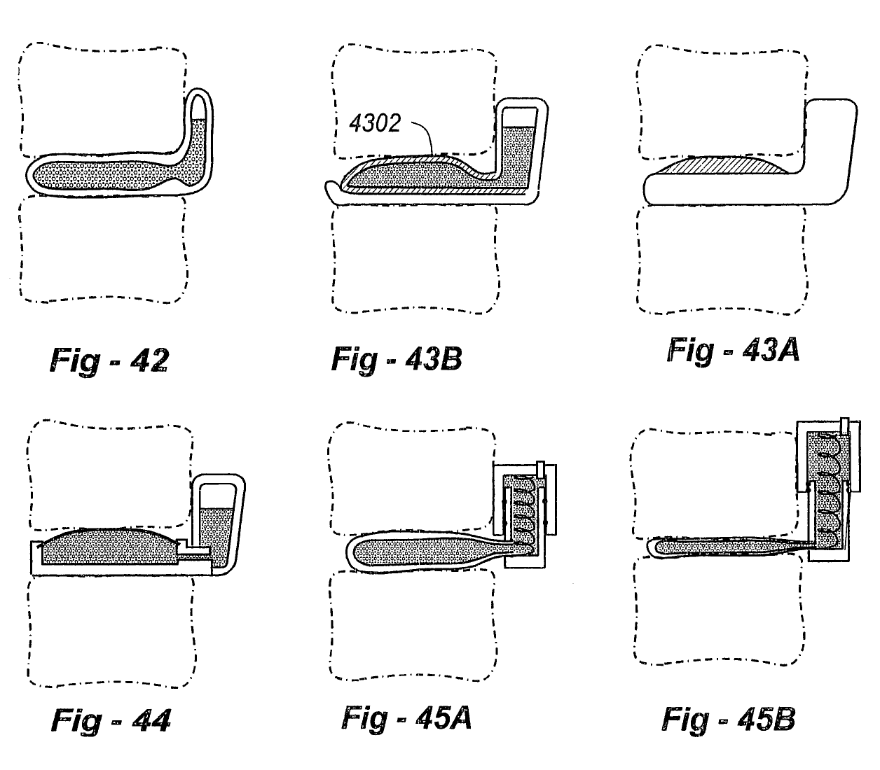

FIG. 42 is a coronal cross section of the spine and another embodiment of the device. The device can be filled with a fluid and air. The area 4202 of the drawing represents fluid. The air in the extradiscal component, being more compressible than liquid, is compressed as fluid moves from the intradiscal component to the extradiscal component. The compressed air forces the fluid to return to the intradiscal component once the pressure on the intradiscal component is relieved.

FIG. 43A is a view of the anterior portion of the spine and another embodiment of the invention wherein an extradiscal component surrounds a portion of the intradiscal component 4302. The intradiscal extension of the extradiscal component helps hold the intradiscal and extradiscal components together. FIG. 43B is a coronal cross section of the spine and the embodiment of the device drawn in FIG. 43A. The intradiscal component is threaded into, or otherwise connected to, the extradiscal component.

FIG. 44 is a coronal cross section of the spine and another embodiment of the invention wherein the intradiscal component is a cylinder with a diaphragm covering a portion of the superior surface of the cylinder.

FIG. 45A is a coronal cross section of the spine and another embodiment of the invention including an expandable extradiscal component. Fluid from the intradiscal component forces the two cylinders of the extradiscal component apart. A spring or elastic bands stretches as the cylinders are forced apart. The spring pulls the cylinders of the extradiscal component together forcing the fluid into the intradiscal component once the pressure on the intradiscal component is relieved. Seals are used between the cylinders of the extradiscal component. A valve is included in the extradiscal component to “bleed” air from the system.

FIG. 45B is a coronal cross section of the spine and the embodiment of the device drawn in FIG. 45A. FIG. 45B illustrates expansion of the extradiscal component and compression of the intradiscal component with axial pressure on the spine or flexion of the spine.

In further alternative embodiments, the extradiscal component could be surrounded by a sleeve to help prevent expansion. As a different option, the device may be constructed of metal with spring like shape memory. In the embodiment shown in FIG. 41A, the device is made of metal or plastic, and may or may not include a bias-ply, radial, or belted construction.

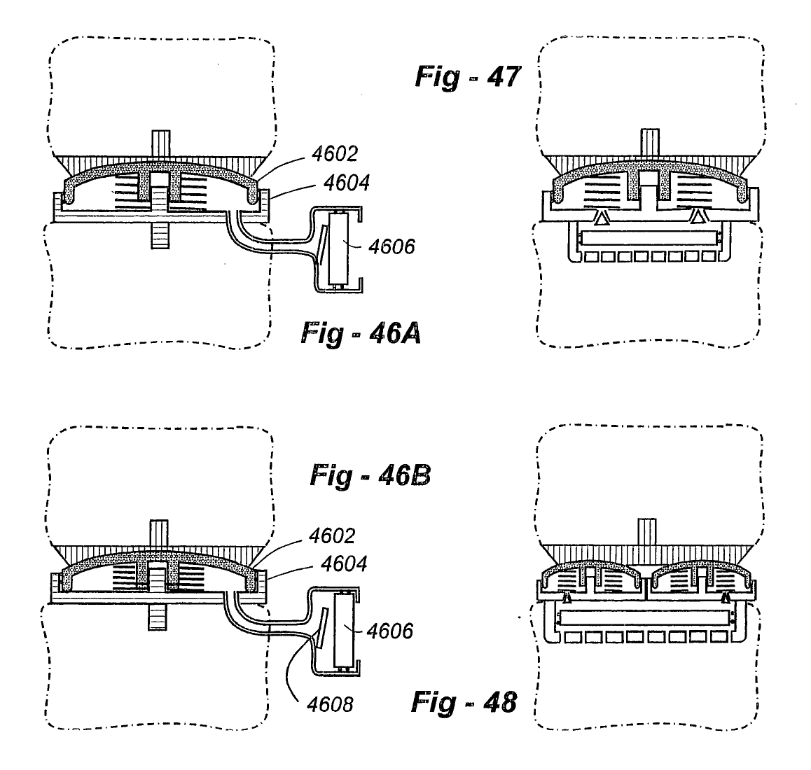

FIG. 46A is a coronal cross section of an alternative embodiment of the invention. The upper ADR Endplate (EP) is represented by the area of the drawing with vertical lines. The upper ADR EP articulates with a second component 4602. For example the two components could articulate through generally concave and generally convex surfaces. The second component also pistons up and down in the lower ADR EP 4604. A spring, or other mechanism such as a wave washer, is used to force the second component from the lower ADR EP.

Seals are preferably used between the second component and the lower ADR EP. For example, O-rings could be used between the components. An extradiscal component is connected to the intradiscal portion of the ADR. The extradiscal component contains a piston 4606, seals, and a valve 4608. The intradiscal component and the extradiscal components contain fluid that freely follows from one component to the other.

FIG. 46B is a coronal cross section of the embodiment of the ADR drawn in FIG. 46A. The drawing illustrates compression of the intradiscal component. Compression of intradiscal component forces fluid to the extradiscal component. The piston of the extradiscal component moves to allow more fluid to enter the extradiscal component. The ADR components return to the positions drawn in FIG. 46A as compression is removed from the intradiscal component. The spring forces the convex intradiscal component away from the lower ADR EP.

The convex intradiscal component has a mechanism to prevent the convex component from disassociating from the lower ADR EP. A piston with elongated arms from the lower ADR EP is inserted through a slot in the cylinder of the convex component. The convex component is then rotated, to couple the two components together. The valve in the extradiscal component dampens the intradiscal component by forcing the fluid to return to the intradiscal component slower than the fluid exited the intradiscal component. A flap valve could be used to slow the fluids return to the intradiscal component. The extradiscal component could be reversibly connected to the intradiscal component to ease the ADR insertion process. The extradiscal component could lie adjacent to the vertebrae. The cylinder of the extradiscal component has extensions to prevent the piston of the extradiscal component from popping out of the ADR.

FIG. 47 is a coronal cross section of an alternative embodiment of the ADR according to the invention wherein an extradiscal component is contained within a vertebra. The drawing also illustrates the use of more than one spring and more than one valve. The valves are represented by the area of the drawing with diagonal lines. The piston of the extradiscal component lies within a chamber that projects from the lower ADR EP, into the lower vertebra. The inferior surface of the chamber has pores to allow natural body fluid to more between the chamber and the vertebra as the piston moves with fluid movement between the intradiscal and extradiscal components. The fluid that moves between the intradiscal component and the extradiscal component is sealed within the components. The fluid sealed within the intradiscal and extradiscal components does not communicate with the fluid than moves into and out of the chamber in the lower vertebra. A seal or seals around the piston of the extradiscal components keeps the two fluids separate.

FIG. 48 is a coronal cross section of an alternative embodiment of the ADR drawn in FIG. 47. The embodiment of the device drawn in FIG. 48 combines the dampening of the invention with the multiple springs and spherical joints taught in my co-pending U.S. patent application Ser. No. 10/434,931 incorporated herein by reference. Fluid from the spring and spherical joint components flows into and out of a single extradiscal component. Multiple extradiscal components could also be used.

As a further option, transplanted cells and/or cells plus the extracellular matrix (ECM) or analogues thereof, may be contained in a device according to the invention. For example, a fluid permeable bag or ‘carcass’ may be used as described in my U.S. Pat. No. 6,419,704, incorporated by reference, or a cylinder or other enclosures as described in my pending U.S. Patent Application Ser. No. 60/379,462, also incorporated by reference, may be used to hold the cells or the cells and ECM within the disc space or elsewhere in the body.

The pores of the device are preferably small enough to prevent cells from leaving or entering the device. Preventing cell migration may help prevent graft vs. host disease. Nutrients and wastes, however, would be free to move through the pores of the device with fluids. The pores of the device could also be large enough for cells to migrate through the pores. The ECM of the transplanted tissue may prevent migration of cells into and out of the device.

The device would also enable intervertebral disc cells to be transplanted to other areas of the body. As described in my co-pending U.S. Patent Application Ser. No. 60/399,597, incorporated herein by reference, the intramedullary canal of long bones and the metaphysis of long bones may be used support the growth of other, non-native, tissues. For example, a cylinder device filled with intervertebral disc cells and ECM, or chondrocytes and ECM could be used to cushion or damper prosthetic joints.

The prosthetic joints could be similar to those disclosed in the pending '597 Application referenced above. Intervertebral disc cells and ECM, as well as, chondrocytes and ECM could also be used to cushion joints without the encapsulating device. The device could also contain stents to enhance circulation, similar to those described in my pending co-pending U.S. patent application Ser. No. 10/143,237, further incorporated herein by reference.

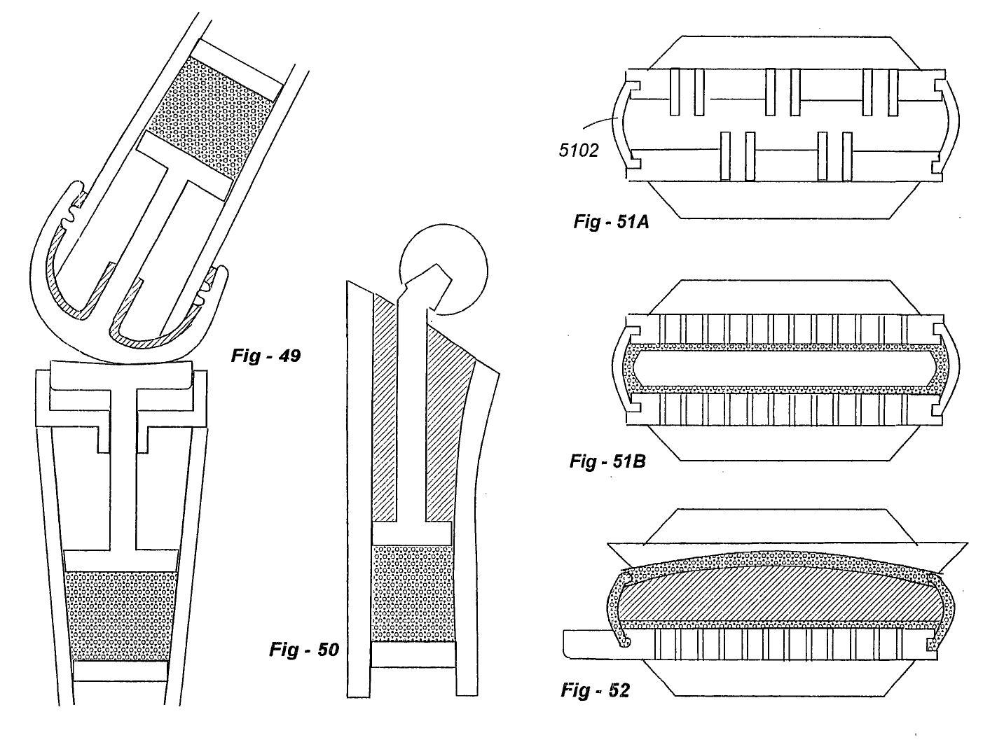

FIG. 49 is a sagittal cross section of a total knee replacement, and FIG. 50 is a sagittal cross section of a total hip replacement. In these embodiments of the invention, IVD cells and ECM and/or chondrocytes and ECM are represented by the dotted area of the drawing. The articular component of the knee replacement is connected to a piston disposed within the cylinder of the device. Cells and cells plus ECM cushion the motion of the knee replacement. The cells and ECM do not necessarily need to be contained within a cylinder device. For example, the cells and ECM could sit directly above a “cement restrictor-like” device. Polymers, gels, fluids, or elastomers could be used in place of the cells and ECM. Cells have the advantage of self-repair. The piston would have holes if fluid is used in a hydraulic-like shock absorber.

FIG. 51A is a sagittal cross section of a disc embodiment of the invention having superior and inferior endplates that attach to the vertebrae above and below the disc. A flexible membrane 5102 surrounds or encapsulates the disc tissue. Stents (as described in my co-pending U.S. patent application Ser. No. 10/143,237, incorporated herein by reference) can be seen coursing through the artificial endplates. The stents allow nutrition and fluid to pass from the vertebrae to the disc tissue. The opening into the stents could be small enough to prevent cells from migrating into or out of the device. For example, the opening in the stents could be 1-7 micrometers in size. Autograft disc tissue removed from the disc to allow insertion of the device, could be placed into the device as described in my co-pending U.S. patent application Ser. No. 10/120,763, similar to the device described in my co-pending U.S. patent application Ser. No. 10/434,917, both of which are incorporated herein by reference.

FIG. 51B is a sagittal cross section an alternative disc embodiment of the invention. The artificial endplates contain pores to allow fluid to move into and out of the device. A permeable membrane lies between the artificial endplates and the disc tissue. The holes in the membrane are sized to prevent the migration of cells into or out of the device. The holes in the artificial endplates can be larger than seven micrometers. FIG. 52 is a sagittal cross section of an alternative disc embodiment of the invention. The portion of the device that encapsulates the disc tissue articulates with an artificial endplate that attaches to the inferior surface of the superior vertebra.

I claim:

1. A prosthetic implant configured for placement between opposing bones that apply pressure to the implant during articulation, the implant comprising:
a fluid-filled reservoir; and
a body coupled to at least one of the bones and the reservoir to provide cushioning during articulation.

2. The prosthetic implant of claim 1, wherein the body is a piston.

3. The prosthetic implant of claim 1, wherein the fluid is water or an aqueous solution.

4. The prosthetic implant of claim 1, wherein the fluid is a synthetic or naturally occurring oil.

5. The prosthetic implant of claim 1, wherein the fluid is an organic or inorganic oil.

6. The prosthetic implant of claim 1, further including superior and inferior endplates; and
wherein the body is coupled to at least one of the endplates as part of an intervertebral disc replacement.

7. The prosthetic implant of claim 1, further including a proximal tibial component and a distal femoral component; and
wherein the body is coupled to at least one of the proximal tibial and distal femoral components as part of a total knee replacement.

8. The prosthetic implant of claim 1, further including an acetabular component and a proximal femoral component; and
wherein the body is coupled to at least one of the acetabular and proximal femoral components as part of a total hip replacement.

9. The prosthetic implant of claim 1, further including a valve or other device that allows the fluid to be expelled from the reservoir during the application of the pressure and drawn back into to the reservoir as the pressure is relieved.

10. The prosthetic implant of claim 1, wherein the fluid is expelled relatively rapidly from the reservoir during the application of pressure, and drawn back into the reservoir at a relatively slow rate as the pressure is relieved.

11. The prosthetic implant of claim 1, further including one or more springs to assist in moving the body from the reservoir as pressure is relieved.

12. The prosthetic implant of claim 1, further including a membrane to contain debris or particulates.

13. The prosthetic implant of claim 1, further including multiple reservoirs, each with a body coupled to one of the bones.

14. The prosthetic implant of claim 1, further including a wheel or other rolling component to control articulation.

15. The prosthetic implant of claim 1, further including a prosthetic femoral head to which the body is coupled.

16. The prosthetic implant of claim 1, wherein the fluid-filled reservoir is associated with an intramedullary stem.

17. The prosthetic implant of claim 1, wherein:
the fluid-filled reservoir is disposed between the opposing bones and further including a second reservoir not disposed between the opposing bones; and
fluid is transferred from the fluid-filled reservoir to the second reservoir when pressure is applied and returned to the fluid-filled reservoir when pressure is relieved.

18. The prosthetic implant of claim 1, wherein the fluid in the reservoir includes one or more biologic constituents.

19. The prosthetic implant of claim 18, wherein the biologic constituents include intervertebral disc cells.

20. The prosthetic implant of claim 18, wherein the biologic constituents include an extracellular matrix or analogues thereof.

21. The prosthetic implant of claim 1, further including a fluid permeable membrane having pores small enough to prevent cell migration while facilitating the transfer of nutrients and/or waste materials.

22. The prosthetic implant of claim 1, wherein the fluid-filled reservoir or other components may be customized to suit a patent's weight or activity level.

23. The prosthetic implant of claim 1, further including a return spring having a stiffness selected to suit a patient's weight or activity level.
